(19) United States
(12) Patent Application Publication
(10) Pub. No.: US 2024/0023923 A1
(43) Pub. Date: Jan. 25, 2024

(54) SONOLUCENT CRANIAL RECONSTRUCTION DEVICE

(71) Applicant: Longeviti Neuro Solutions LLC, Hunt Valley, MD (US)

(72) Inventors: Corbin Clawson, Hampstead, MD (US); Bradley Rabinovitz, Severna Park, MD (US)

(21) Appl. No.: 18/355,941

(22) Filed: Jul. 20, 2023

Related U.S. Application Data
(60) Provisional application No. 63/369,014, filed on Jul. 21, 2022.

(51) Int. Cl. A61B 8/08; A61M 37/00; A61B 17/02

(52) U.S. Cl. CPC A61B 8/0808; A61M 37/0092; A61B 8/481; A61B 17/0218; A61M 2210/0693

(57) ABSTRACT

A sonolucent cranial reconstruction device is optimized for trans-cranioplasty ultrasound. The cranial reconstruction device includes a tubular base member and a sonolucent upper wall extending across the top of the tubular base member. The tubular base member is shaped and dimensioned for positioning within an aperture defined by a cranial defect such that bone growth is prevented within the aperture defined by the cranial defect.

### CROSS REFERENCE TO RELATED APPLICATION

This application claims the benefit of U.S. Provisional Patent Application Ser. No. 63/369,014, entitled “SONOLUCENT CRANIAL RECONSTRUCTION DEVICE,” filed Jul. 21, 2022, which is incorporated herein.

### BACKGROUND OF THE INVENTION

### 1. Field of the Invention

The invention relates to cranial reconstruction devices.

### 2. Description of the Related Art

Bone remodeling takes old and damaged bone and replaces with new bone. The process involves both bone resorption and bone formation. Bone formation results in additional bone growth. It is known that long bones rely almost exclusively on mechanical forces to trigger bone remodeling (Wolf's Law). However, flat bones, such as the skull vault, are formed by intramembranous ossification where mesenchymal cells differentiate into osteoblasts which secrete and mineralize osteoid directly to form plate-like bones, also known as, metabolic bone remodeling. Kenkre J S, Bassett J. The bone remodelling cycle. Ann Clin Biochem. 2018 May;55(3):308-327. doi: 10.1177/0004563218759371. Epub 2018 Mar. 4. PMID: 29368538.

In small cranial defects, the bone remodels via this metabolic pathway and eventually occludes the defect through the bone growth. Such remodeling is known to occur in existing cranial devices that are not as thick as the cranium and therefore allow for bone remodeling, including bone growth, to occur distal to the implant/proximal to the dura.

### SUMMARY OF THE INVENTION

According to one aspect a sonolucent cranial reconstruction device is optimized for trans-cranioplasty ultrasound. The cranial reconstruction device includes a tubular base member and a sonolucent upper wall extending across a top of the tubular base member. The tubular base member is shaped and dimensioned for positioning within an aperture defined by a cranial defect such that bone growth is prevented within the aperture defined by the cranial defect.

In some embodiments the tubular base member extends a thickness of a cranium to mechanically prevent bone remodeling in the cranial defect, thereby preserving acoustic pass-through.

In some embodiments the tubular base member is formed with a cross sectional profile selected from the group consisting of cylindrical, square, rectangular, oval, and polyhedral.

In some embodiments the tubular base member includes an annular frame member defining an outer perimeter of the tubular base member, the annular frame member includes an upper surface, a lower surface, an inner sidewall extending between the upper surface and the lower surface along a cavity defined by an interior of the annular frame member, and an outer sidewall extending between the upper surface and the lower surface of the annular frame member.

In some embodiments the inner sidewall and the outer sidewall define consistent and continuous diameters.

In some embodiments the tubular base member is 7 mm to 9 mm from the upper surface to the lower surface.

In some embodiments the sonolucent upper wall is coextensive with the upper surface of the annular frame member such that the sonolucent upper wall and the inner sidewall define a cavity of the cranial reconstruction device.

In some embodiments a thickness of the sonolucent upper wall is less than a length of the tubular base member, thus forming the cavity defining an open area inside the tubular base member.

In some embodiments a space defined by the cavity is filled with a highly ultrasound transmissive fluid, gel, or other material to enhance or otherwise control passage of ultrasound waves through the cranial reconstruction device.

In some embodiments a lensing window is integrated into the cavity of the tubular base member to manipulate or alter ultrasound wave.

In some embodiments the sonolucent upper wall includes an upper surface, a lower surface, and a sidewall extending between the upper surface and the lower surface.

In some embodiments the sidewall of the sonolucent upper wall is tapered inwardly along a lower portion thereof such that a diameter of the sidewall decreases as it extends from the upper surface toward the lower surface of the sonolucent upper wall.

In some embodiments the sonolucent upper wall is further provided with outwardly extending tab members coextensive with the sonolucent upper wall.

In some embodiments the tubular base member and the sonolucent upper wall are integrally formed.

In some embodiments the tubular base member and the sonolucent upper wall are manufactured from sonolucent poly (methyl methacrylate) (PMMA).

In some embodiments the sonolucent upper wall and the tubular base member are distinct pieces that are selectively assembled and/or disassembled.

In some embodiments the tubular base member and the sonolucent upper wall are formed from different materials offering optimal physical characteristics for their intended purposes.

In some embodiments the tubular base member is made of porous polyethylene.

In some embodiments the sonolucent upper wall is made of PMMA.

In some embodiments an ultrasound transducer is further included.

In another aspect a method for preventing bone growth within a cranial defect includes forming a cranial defect and implanting a cranial reconstruction device within the cranial defect such that the cranial reconstruction device covers the entire internal surface of the cranial defect.

In some embodiments of this method the cranial reconstruction device is sonolucent.

In some embodiments of this method a step of passing ultrasound through the cranial reconstruction device is further included.

In some embodiments of this method the cranial reconstruction device includes a tubular base member and a sonolucent upper wall extending across a top of the tubular base member.

In some embodiments of this method a step of acting upon and/or monitoring a contrast agent as it travels within the brain is further included.

In some embodiments of this method a step of imaging confirmation of blood flow immediately after surgery, imaging confirmation of blood flow during follow-up examinations, imaging confirmation of blood flow to desired locations within the brain, imaging visualization of, and guidance during, interventional procedures to guide a wire and/or catheter, or activating a contrast agent as it passes a particular point of interest and is subjected to ultrasound for activation thereof is further included.

In some embodiments of this method a step of utilizing ultrasound in conjunction with drug delivery is further included.

In some embodiments of this method a step of subjecting a drug to ultrasound at a specific location within the brain so that the drug is active at a highly specific location, subjecting a drug to ultrasound at a specific location within the brain to enhance effectiveness of the drug through delivery of ultrasound, subjecting the drug to ultrasound at a specific location within the brain to assist the drug in passing through the brain blood barrier, or therapeutically releasing nano-encapsulated drugs through the application of ultrasound wherein the ultrasound breaks down the encapsulation material to release the drug is further included.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a front elevation view of a cranial reconstruction devices installed within a cranium.

FIG. 2 is a perspective view of the cranial reconstruction devices.

FIG. 3 is a cross section view of the cranial reconstruction devices shown in FIG. 2.

FIG. 4 is an exploded view of the cranial reconstruction devices being implanted within the cranium.

FIGS. 5, 6, and 7 disclose the sequence of steps in the installation and use of the cranial reconstruction devices.

FIG. 8 is a cross section view of the cranial reconstruction devices implanted within the cranium.

FIGS. 9 and 10 are cross section views of alternate embodiments of the cranial reconstruction devices.

FIG. 11 is a perspective view of an alternate embodiment of the cranial reconstruction devices.

FIG. 12 is a cross section view of the cranial reconstruction device shown in FIG. 11.

FIG. 13 is a perspective view of a cranial reconstruction device, with hidden (broken) lines showing hidden contours, in accordance with another embodiment.

FIG. 14 is a perspective view of the cranial reconstruction device shown in FIG. 13, with the sonolucent upper wall removed.

FIG. 15 is a cross sectional view of the cranial reconstruction device shown in FIG. 13.

FIG. 16 is a side elevation view of the cranial reconstruction device shown in FIG. 13.

FIG. 17 is a top plan view of the cranial reconstruction device shown in FIG. 13.

FIG. 18 is a top plan view of a cranial reconstruction device in accordance with another embodiment.

FIG. 19 is a perspective view of the cranial reconstruction device shown in FIG. 18.

FIG. 20 is a cross sectional view of the cranial reconstruction device shown in FIG. 18.

### DESCRIPTION OF THE EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

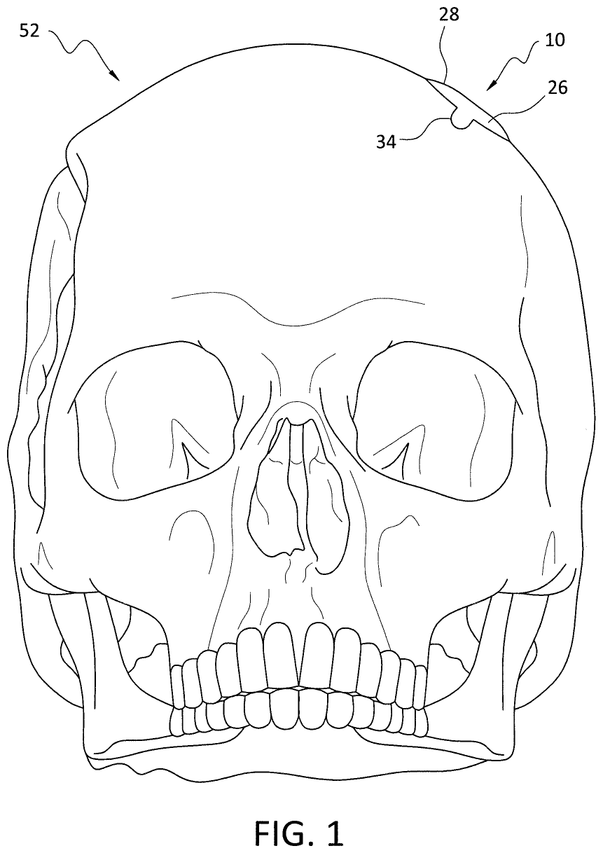

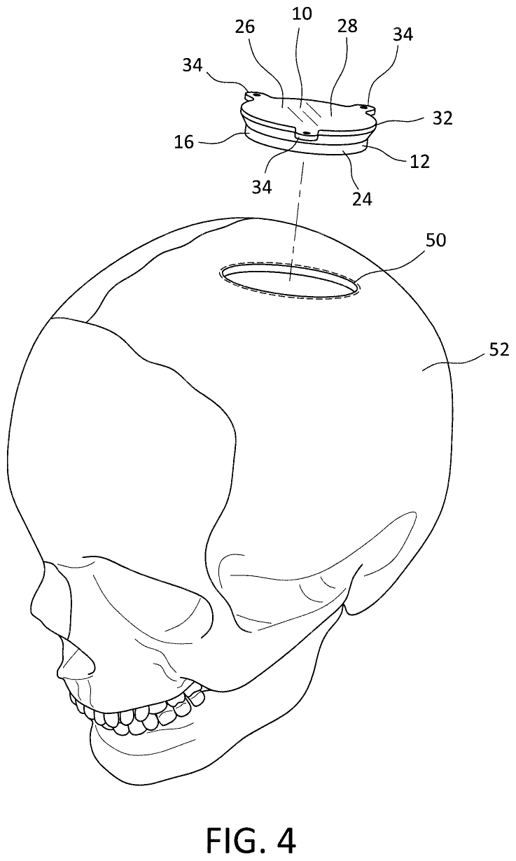

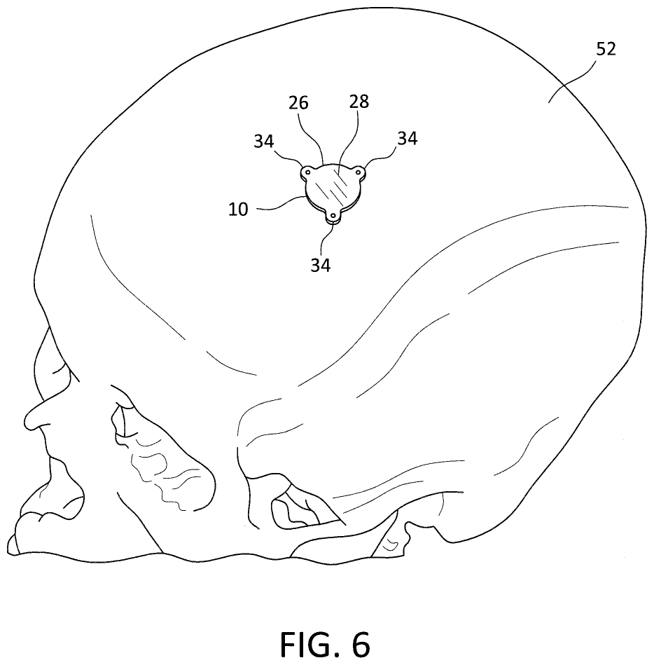

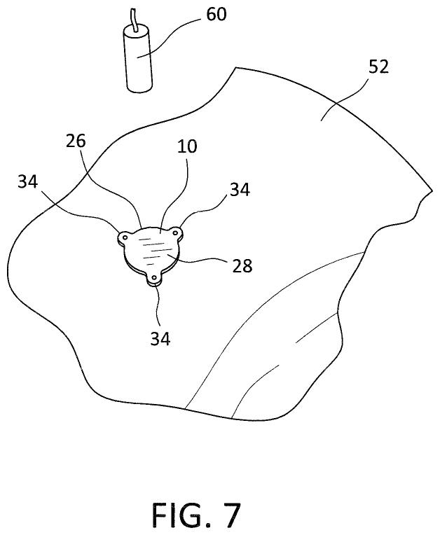

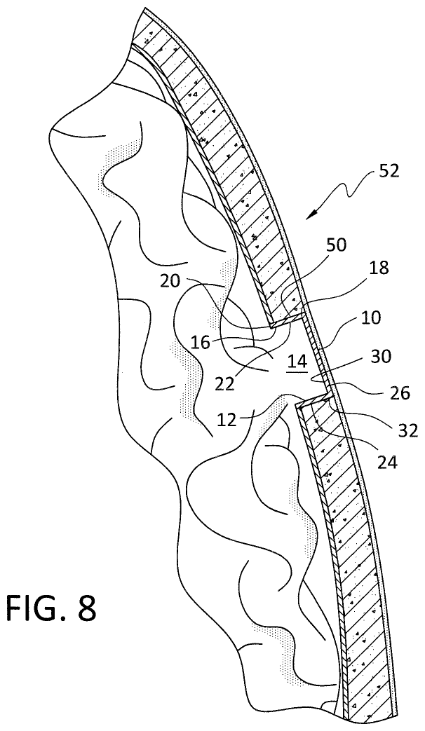

Referring to FIGS. 1 to 8, a sonolucent cranial reconstruction device 10 is disclosed. As will be appreciated based upon the following detailed disclosure, the sonolucent cranial reconstruction device 10 includes a tubular base member 12 shaped and dimensioned for positioning within the aperture defined by the cranial defect 50 such that bone growth is prevented within the aperture defined by the cranial defect 50 and a sonolucent upper wall 26 optimized for trans-cranioplasty ultrasound. It should be understood that the term sonolucent is meant to refer to materials allowing the passage of ultrasonic waves without production of echoes that are due to reflection of some of the waves. The cranial reconstruction device 10 functions to reconstruct the cranium 52 after a cranial defect 50 is made for intracranial access, enable post-operative trans-cranioplasty ultrasound, and prevent the cranium from regrowing bone that would inhibit long term post-operative imaging via ultrasound.

As discussed above, bone remodeling may occur in conjunction with cranial devices. This bone remodeling, and ultimately any impediments to the use of the defect for trans-cranioplasty ultrasound, is obviated by the present cranial reconstruction device 10 that extends the thickness of the cranium to mechanically prevent bone remodeling in the cranial defect 50 and preserve the acoustic pass-through provided by the cranial defect. The present cranial reconstruction device 10 includes a tubular base member 12 that extends the thickness of the cranium 52 to mechanically prevent bone remodeling in the cranial defect; thereby preserving the acoustic pass-through.

The present cranial reconstruction device 10 is preferably shaped and dimensioned for positioning within a burr hole, cranial access point, or other cranial defect 50 made in the cranium 52 of a patient. As those skilled in the art will appreciate, cranial plugs (commonly referred to as burr hole plugs) are used in conjunction with a variety of surgical procedures requiring access to the brain. The procedures generally require the formation of a “burr hole” within the cranium. The burr hole a lows surgeons to access the brain for the desired procedure. For example, burr holes are commonly used in conjunction with deep brain stimulation procedures where a stimulation lead is implanted within the brain to position electrodes adjacent predetermined tissue of the brain. The electrodes are then used to electrically stimulate the tissue of the brain for treatment of a specific disease or disorder.

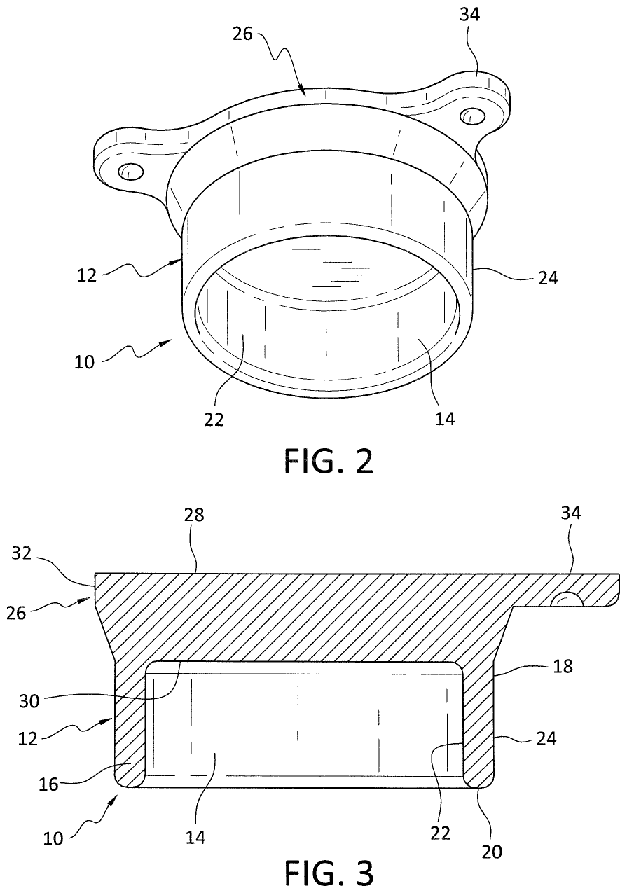

The present cranial reconstruction device 10 provides a convenient, reliable, and effective solution to both covering the cranial defect 50 and maintaining a window for viewing of the neuroanatomy via ultrasound. Referring to FIGS. 2 to 3, and as briefly mentioned above, the cranial reconstruction device 10 includes a tubular base member 12 shaped and dimensioned for positioning withing the aperture defined by the cranial defect 50. It should also be appreciated that the tubular base member 12 may be formed with a variety of cross sectional profiles, including, but not limited to, cylindrical, square, rectangular, oval, polyhedral, or other shapes conforming to the desired/created opening shape.

The tubular base member 12 is substantially cylindrical and includes an annular frame member 16 defining the outer perimeter of the tubular base member 12. The annular frame member 16 includes an upper surface 18, a lower surface 20, an inner sidewall 22 extending between the upper surface 18 and the lower surface 20 along the cavity 14 defined by the interior of the annular frame member 16, and an outer sidewall 24 extending between the upper surface 18 and the lower surface 20 of the annular frame member 16. In accordance with a disclosed embodiment, the inner and outer sidewalls 22, 24 define consistent and continuous diameters.

As discussed above, the cranial reconstruction device 10 extends the thickness of the cranium 52 to mechanically prevent bone remodeling in the cranial defect 50 and preserve the acoustic pass-through provided by the cranial defect 50. As a result, the tubular base member 12 extends the thickness of the cranium to mechanically prevent bone remodeling in the cranial defect 50 and is accordingly 7 mm to 9 mm from the upper surface 18 to the lower surface 20 in order to accommodate most full thickness defects without significantly protruding towards the cortex or scalp.

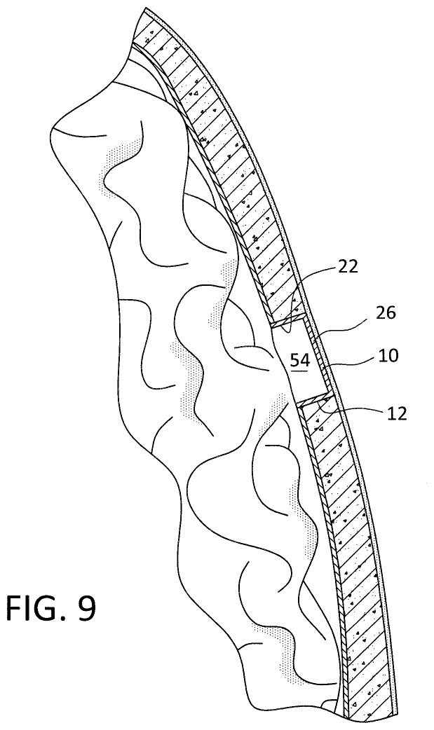

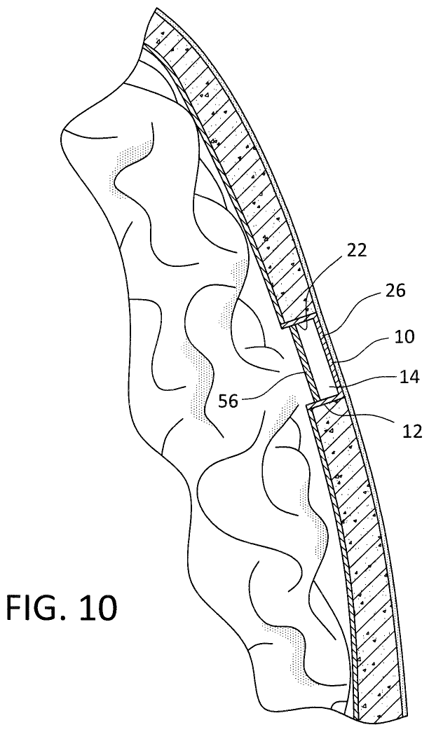

The cranial reconstruction device 10 further includes a sonolucent upper wall 26 extending across the top of the tubular base member 12 and being coextensive with the upper surface 18 of the annular frame member 16. As such, the sonolucent upper wall 26 and the inner sidewall 22 define a cavity 14 of the cranial reconstruction device 10. With this in mind, it is appreciated that the thickness of the upper wall is less than the length of the tubular base member 12 (that is, the distance from the upper surface 18 of the tubular base member 12 to the lower surface 20 of the tubular base member 12), thus forming the cavity 14 defining an open area inside the tubular base member. In accordance with a disclosed embodiment, the cavity 14 fills with cerebral spinal fluid, which assists in ensuring sonic coupling between the ultrasound transducer 60 and the neuroanatomy of interest. It is further contemplated in accordance with an alternate embodiment that the space defined by the cavity 14 could be filled with a highly ultrasound transmissive fluid, gel, or other material 54 to enhance or otherwise control the passage of the ultrasound waves through the cranial reconstruction device 10 to assure coupling of the ultrasound signal into the brain (see FIG. 9 where similar reference numerals are used for parts similar to those disclosed with reference to the embodiment in FIGS. 1 to 8). In accordance with a further embodiment, a lensing window 56 could be integrated into the cavity 14 of the tubular base member 12 so as to manipulate or alter the ultrasound wave to enhance or otherwise control the passage of the ultrasound waves through the cranial reconstruction device 10 to assure coupling of the ultrasound signal into the brain (see FIG. 10 where similar reference numerals are used for parts similar to those disclosed with reference to the embodiment in FIGS. 1 to 8). Where a filling material 54 is used within the cavity 14 of the tubular base member 12, it is contemplated tissue granulation would be minimized thereby likely improving the long-term functioning of the cranial reconstruction device 10.

The sonolucent upper wall 26 is substantially disc shaped and includes an upper surface 28, a lower surface 30, and a sidewall 32 extending between the upper surface 28 and the lower surface 30. As will be appreciated based upon the following disclosure regarding optimizing ultrasound transmission through the sonolucent upper wall 26 based upon consideration of attenuation and other parameters, the thickness and material of the sonolucent upper wall 26 are optimized for the type of ultrasound transmission that is anticipated or desired. Briefly, attenuation is measured in sound loss per centimeter. Therefore, thinner is generally better as less attenuation of the sound waves passing through the sonolucent upper wall 26 occurs. However, it is contemplated adjustments and additions (for example, the cavity 14 might fill with granulation tissue) to the cavity 14 might make it desirable to increase the thickness of the sonolucent upper wall 26. In accordance with one embodiment, the sonolucent upper wall 26 is constructed with a thickness of less than 3.5 mm.

In accordance with a disclosed embodiment, the sidewall 32 of the sonolucent upper wall 26 is tapered inwardly along the lower portion thereof such that the diameter of the sidewall 32 decreases as it extends from the upper surface 28 toward the lower surface 30 of the sonolucent upper wall 26. This taper helps to guide the cranial reconstruction device 10 into the cranial defect 50 as it is pressed into the cranial defect 50. As discussed below in greater detail, it should be appreciated that the cranial reconstruction device 10 is provided with tab members 34 that sit on top of the cranium. However, it is appreciated some surgeons may use available cutting accessories to recess the tab members 34 into the outer cortex.

The cranial reconstruction device 10, and in particular the sonolucent upper wall 26, is further provided with outwardly extending tab members 34 coextensive with the sonolucent upper wall 26. The outwardly extending tab members 34 prevent the cranial reconstruction device 10 from “plunging” into the cranial defect 50 in the event the cranial defect 50 is too large for the cranial reconstruction device 10 or if inadvertent force is applied to the cranial reconstruction device 10 causing it to move through the cranial defect 50 and into the intracranial space. The tab members 34 further allow for the passage of bone screws therethrough and allow for secure attachment of the cranial reconstruction device 10 to the cranium 52.

The tubular base member 12 and the sonolucent upper wall 26 are preferably integrally formed and are manufactured from sonolucent poly (methyl methacrylate) (PMMA) or any other sonolucent biocompatible materials suited for safe use in craniofacial reconstruction. Regardless of whether the tubular base member 12 and the sonolucent upper wall 26 are constructed from PMMA, the sonolucent upper wall 26 is constructed of material(s) having “sonolucent properties” exhibiting low enough attenuation to enable ultrasound imaging as an acoustic pass-through (for example PEEK (polyetheretherketone) could be used as a replacement for PMMA as it is known to exhibit similar sonolucent characteristics. While PMMA is used in accordance with a disclosed embodiment as discussed herein, it is appreciated the cranial reconstruction device 10 may include a polymer, metal, bioengineered material, or any combinations thereof. Further still, the tubular base member 12 and the sonolucent upper wall 26 can be made of different materials, as the tubular base member 12 need not be sonolucent whereas the sonolucent upper wall 26 should be sonolucent.

By way of example, the cranial reconstruction device 10 may be manufactured in a manner allowing for the transmission of ultrasonic waves as described in U.S. Pat. No. 9,044,195, entitled “IMPLANTABLE SONIC WINDOW,” ('195 Patent) which is incorporated herein by reference. As explained in the '195 Patent, a strong, porous sonically translucent material through which ultrasonic waves can pass for purposes of imaging the brain is employed, wherein the material is a polymeric material, such as polyethylene, polystyrene, acrylic, or poly(methyl methacrylate) (PMMA). In addition, U.S. Pat. No. 9,535,192, entitled “METHOD OF MAKING WAVEGUIDE-LIKE STRUCTURES,” ('192 Patent) and U.S. Patent Application Publication No. 2017/0156596, entitled “CRANIAL IMPLANTS FOR LASER IMAGING AND THERAPY,” ('596 Publication) both of which are incorporated herein by reference, disclose making waveguide-like structures within optically transparent materials using femtosecond laser pulses wherein the optically transparent materials are expressly used in the manufacture of cranial implants. The '596 publication explains the use of optically transparent cranial implants and procedures using the implants for the delivery of laser light into shallow and/or deep brain tissue. The administration of the laser light can be used on demand, thus allowing real-time and highly precise visualization and treatment of various pathologies. Further still, Tobias et al. describe an ultrasound window to perform scanned, focused ultrasound hyperthermia treatments of brain tumors. Tobias et al., “ULTRASOUND WINDOW TO PERFORM SCANNED, FOCUSED ULTRASOUND HYPERTHERMIA TREATMENTS OF BRAIN TUMORS,” Med. Phys. 14(2), March/April 1987, 228-234, which is incorporated herein by reference. Tobias et al. tested various materials to determine which material would best serve as an acoustical window in the skull and ultimately determined polyethylene transmitted a larger percentage of power than other plastics and would likely function well as an ultrasonic window. Further still, Fuller et al., “REAL TIME IMAGING WITH THE SONIC WINDOW: A POCKET-SIZED, C-SCAN, MEDICAL ULTRASOUND DEVICE,” IEEE International Ultrasonics Symposium Proceedings, 2009, 196-199, which is incorporated herein by reference, provides information regarding sonic windows.

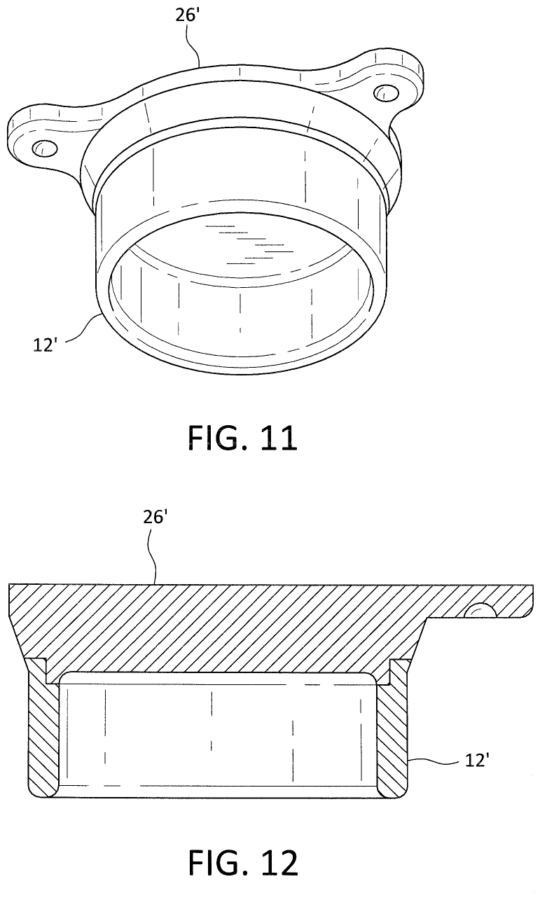

While the sonolucent upper wall 26 and the tubular base member 12 are formed integrally in accordance with a disclosed embodiment, it is appreciated the upper wall 26′ and the tubular base member 12′ could be two distinct pieces that are selectively assembled and/or disassembled (see FIGS. 11 and 12). It is contemplated the tubular base member 12′ and the upper wall 26′ might be formed from different materials offering optimal physical characteristics for their intended purposes; for example, the tubular base member 12′ might be made of porous polyethylene offering ideal physical characteristics for mounting within the cranial opening (but exhibiting poor sonolucent characteristics), the upper wall 26′ would be made of PMMA offering optimal sonolucent characteristics, and the upper wall 26′ would be constructed to frictionally fit within the tubular base member 12′. In accordance with a two-piece embodiment as discussed above, a friction fit between the defect and the base member may be used to hold the base member. Additionally, a set screw, lock in key rotating method, threaded screw & bolt method, etc. could also be use where for additional rigidity is desired and the coupling structure does not interfere with the acoustic window.

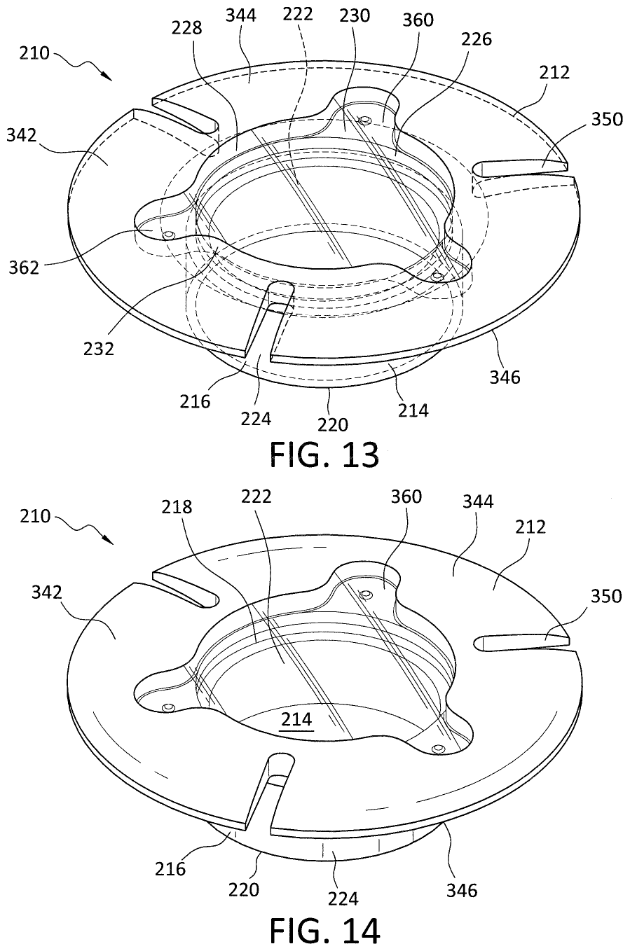

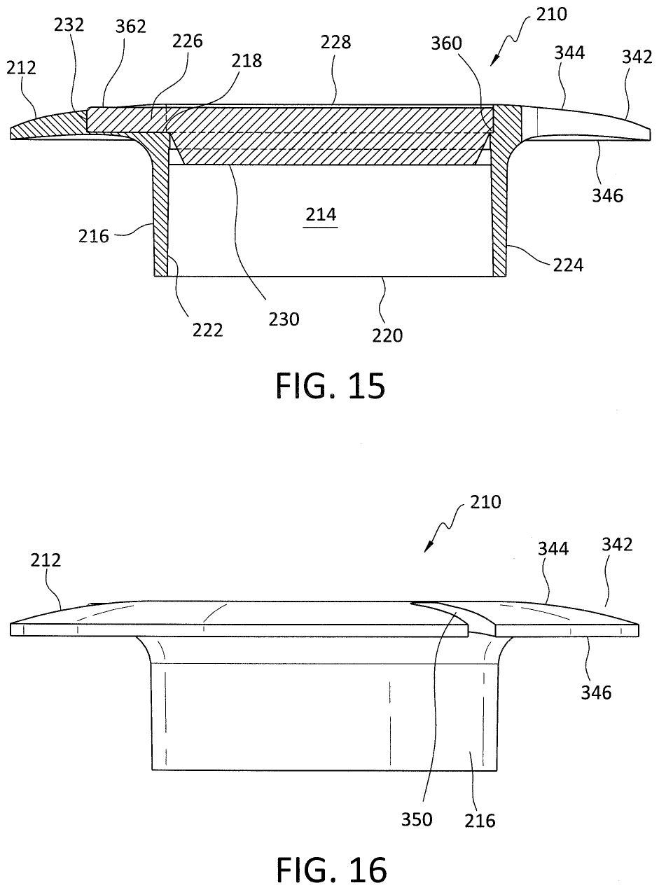

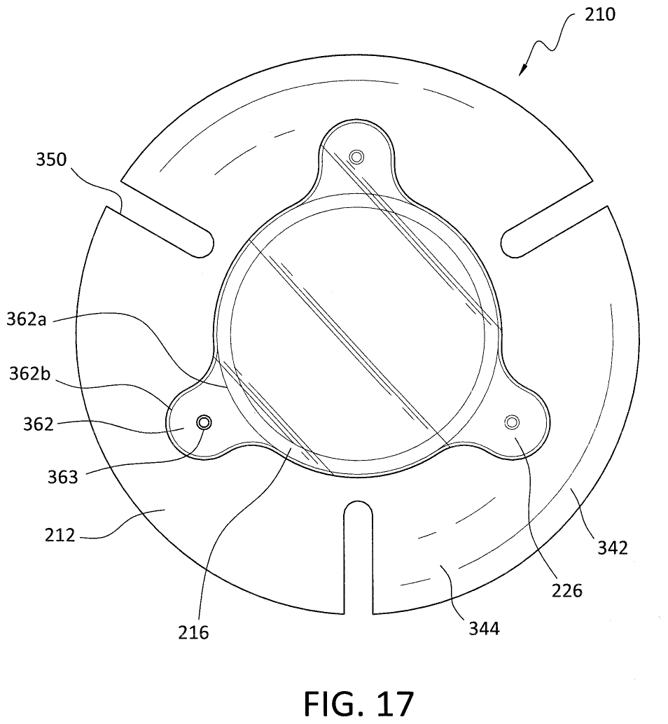

In accordance with another embodiment, as discussed with reference to FIGS. 13 to 17, the sonolucent cranial reconstruction device 210 includes a base member 212 shaped and dimensioned for positioning within the aperture defined by the cranial defect 50 such that bone growth is prevented within the aperture defined by the cranial defect 50 and a sonolucent upper wall 226 optimized for trans-cranioplasty ultrasound. The cranial reconstruction device 210 functions to reconstruct the cranium 52 after a cranial defect 50 is made for intracranial access, enable post-operative trans-cranioplasty ultrasound, and prevent the cranium from regrowing bone that would inhibit long term post-operative imaging via ultrasound.

As with the prior embodiments, the cranial reconstruction device 210 includes a base member 212 that extends the thickness of the cranium 252 to mechanically prevent bone remodeling in the cranial defect; thereby preserving the acoustic pass-through. Bone remodeling, and ultimately any impediments to the use of the defect for trans-cranioplasty ultrasound, are obviated by the present cranial reconstruction device 210, in particular, the base member 212, that extends the thickness of the cranium to mechanically prevent bone remodeling in the cranial defect 50 and preserve the acoustic pass-through provided by the cranial defect.

The present cranial reconstruction device 210 is preferably shaped and dimensioned for positioning within a burr hole, cranial access point, or other cranial defect 50 made in the cranium 52 of a patient. Referring to FIGS. 13 to 17, and as briefly mentioned above, the cranial reconstruction device 210 includes a base member 212 shaped and dimensioned for positioning withing the aperture defined by the cranial defect 50. As with the prior embodiments, the base member 212 may be formed with a variety of cross sectional profiles, including, but not limited to, cylindrical, square, rectangular, oval, polyhedral, or other shapes conforming to the desired/created opening shape.

The base member 212 is substantially cylindrical and includes an annular frame member 216 defining the outer perimeter of that portion of the base member 212 sitting within the cranial defect 50. The annular frame member 216 includes an upper surface 218, a lower surface 220, an inner sidewall 222 extending between the upper surface 218 and the lower surface 220 along the cavity 214 defined by the interior of the annular frame member 216, and an outer sidewall 224 extending between the upper surface 218 and the lower surface 220 of the annular frame member 216. In accordance with a disclosed embodiment, the inner sidewall 222 defines a consistent and continuous diameter, while the outer side wall 224 defines a consistent and continuous diameter along the lower portions thereof but tapers outwardly slightly as it approaches the upper surface 218.

Extending radially outwardly from the upper surface 218 of the annular frame member 216 is an annular flange 342. The annular flange 342 extends outwardly from the upper surface 218 of the annular frame member 216. The flange 342 includes an upper surface 344 that defines the exterior first surface and a lower surface 346 that ultimately is positioned upon the cranium when the base member 212 is positioned in the resected portion of the cranium created during the installation process. The annular flange 342 supports the base member 212 and prevents the base member 212 from going too deep into the resected portion of the cranium, acts as a fixation point to secure the base member 212 to the skull, and, if the resected portion of the cranium is bigger than the base member 212, the annular flange 342 helps cover any spaces between the base member 212 and the cranium.

The flange 342 is further provided with radially extending slots 350 formed along the periphery thereof of the annular frame member 216 and the flange 342. The radially extending slots 350 also act as relief cuts and allow the flange 342 to be bent and conformed to the skull's surface. Without the slots 350, it would be more difficult to bend the flange 342, and the flange 342 may experience rippling if there aren't any relief cuts.

The upper surfaces 218, 344 of the annular frame member 216 and the flange 342, respectively, define a recessed area 360 at the intersection the annular frame member 216 and the flange 342 shaped and dimensioned for receiving the sonolucent upper wall 226. The recessed area 360 is specifically shaped and dimensioned to be wide enough to accept a neurosurgical screw that is used to secure the sonolucent upper wall 226 to the base member 212.

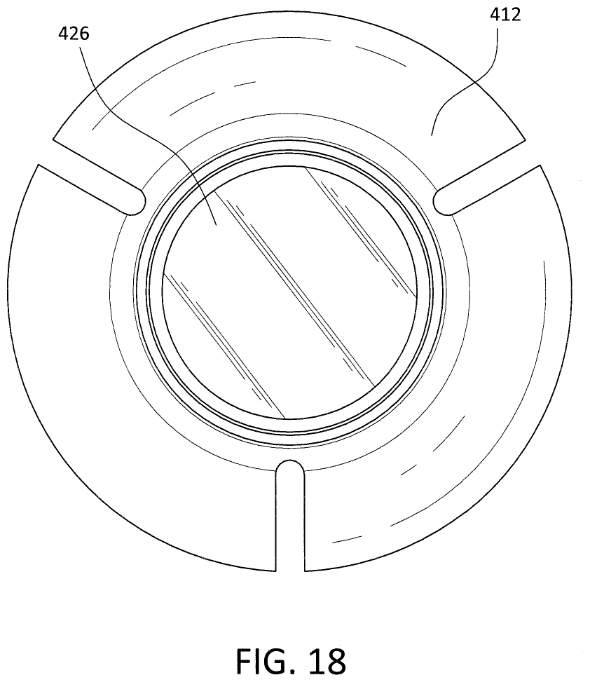

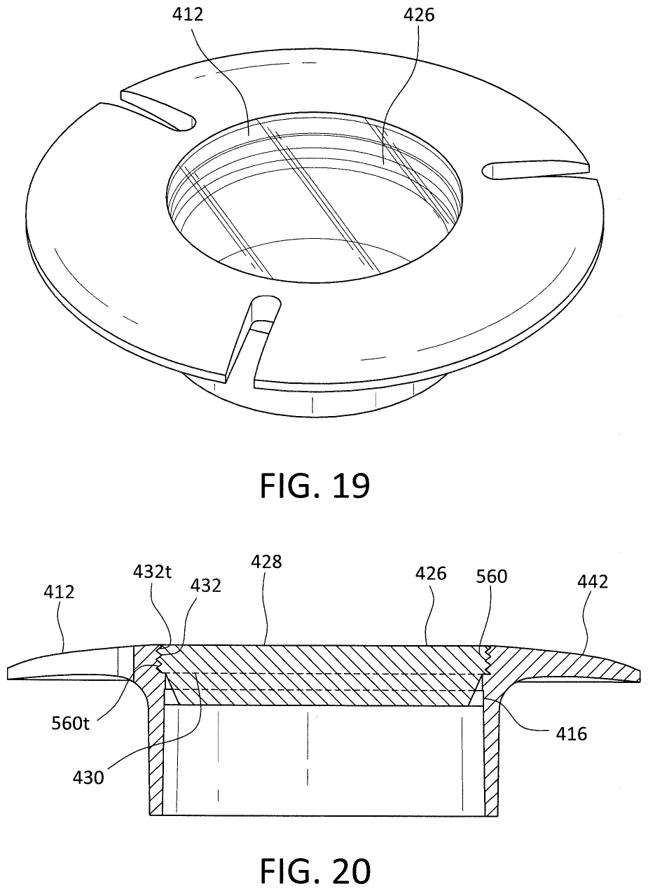

While a screw is disclosed above, it is appreciated the sonolucent upper wall 226 and the base member 212 may be held together by any known mechanisms, including, but not limited to friction fit, glue, screws, snap-fit, etc. In these situations, a disc shaped sonolucent upper wall 426 is desired as it facilitates connections (see FIGS. 18 to 20 as discussed below in more detail).

As discussed above, the cranial reconstruction device 210 extends the thickness of the cranium 52 to mechanically prevent bone remodeling in the cranial defect 50 and preserve the acoustic pass-through provided by the cranial defect 50. As a result, the base member 212 extends the thickness of the cranium to mechanically prevent bone remodeling in the cranial defect 50 and is accordingly 7 mm to 9 mm from the upper surface 218 to the lower surface 220 in order to accommodate most full thickness defects without significantly protruding towards the cortex or scalp.

The cranial reconstruction device 210 further includes a sonolucent upper wall 226 extending across the top of the base member 212 and being coextensive with the upper surface 344 of the flange 342. As such, the sonolucent upper wall 226 and the inner sidewall 222 define a cavity 214 of the cranial reconstruction device 210. With this in mind, it is appreciated that the thickness of the sonolucent upper wall 226 is less than the length of the base member 212 (that is, the distance from the upper surface 218 of the base member 212 to the lower surface 220 of the base member 212), thus forming the cavity 214 defining an open area inside the base member 212.

The sonolucent upper wall 226 is substantially disc shaped and includes an upper surface 228, a lower surface 230, and An exterior side wall 232 extending between the upper surface 228 and the lower surface 230. As will be appreciated based upon the following disclosure regarding optimizing ultrasound transmission based upon consideration of attenuation and other parameters, the thickness and material of the sonolucent upper wall 226 are optimized for the type of ultrasound transmission that is anticipated or desired. Briefly, attenuation is measured in sound loss per centimeter. Therefore, thinner is generally better as less attenuation of the sound waves passing through the sonolucent upper wall 226 occurs. However, it is contemplated adjustments and additions (for example, the cavity 214 might fill with granulation tissue) to the cavity 214 might make it desirable to increase the thickness of the sonolucent upper wall 226. In accordance with one embodiment, the sonolucent upper wall 226 is constructed with a thickness of less than 3.5 mm.

In accordance with a disclosed embodiment, the sonolucent upper wall 226 includes a plurality of coupling arms 362 radially extending outwardly from the exterior side wall 232. In accordance with a disclosed embodiment, the sonolucent upper wall 226 includes three coupling arms 362. Each of the coupling arms 362 includes first end 362a coupled to the central portion of the sonolucent upper wall 226 and a second end 362b positioned beyond the circumference defined by the central portion of the sonolucent upper wall 226. Each of the second ends 362b is provided with a coupling aperture 363 for attaching the sonolucent upper wall 226 to the flange 342.

The base member 212 and the sonolucent upper wall 226 are separate and distinct elements and may be manufactured from different materials to suit specific needs. For example, both elements could be made from sonolucent poly (methyl methacrylate) (PMMA) or any other sonolucent biocompatible materials suited for safe use in craniofacial reconstruction. Regardless of whether the base member 212 and the sonolucent upper wall 226 are constructed from PMMA, the sonolucent upper wall 226 is constructed of material(s) having “sonolucent properties” exhibiting low enough attenuation to enable ultrasound imaging as an acoustic pass-through (for example PEEK (polyetheretherketone) could be used as a replacement for PMMA as it is known to exhibit similar sonolucent characteristics. While PMMA is used in accordance with a disclosed embodiment as discussed herein, it is appreciated the cranial reconstruction device 210 may include a polymer, metal, bioengineered material, or any combinations thereof. Further still, the base member 212 and the sonolucent upper wall 226 can be made of different materials, as the base member 212 need not be sonolucent whereas the sonolucent upper wall 226 should be sonolucent.

It is contemplated the base member 212 and the sonolucent upper wall 226 might be formed from different materials offering optimal physical characteristics for their intended purposes; for example, the base member 212 might be made of porous polyethylene offering ideal physical characteristics for mounting within the cranial opening (but exhibiting poor sonolucent characteristics), the sonolucent upper wall 226 would be made of PMMA offering optimal sonolucent characteristics, and the sonolucent upper wall 226 would be constructed to frictionally fit within the base member 212.

In accordance with a two-piece embodiment as discussed above, a friction fit between the defect and the base member may be used to hold the base member. Additionally, a set screw, lock in key rotating method, threaded screw & bolt method, etc. could also be use where for additional rigidity is desired and the coupling structure does not interfere with the acoustic window.

By way of example, and considering a threaded connection between the sonolucent upper wall 426 and the base member 412, the sonolucent upper wall 426 is disc shaped and includes an upper surface 428, a lower surface 430, and an exterior side wall 432 extending between the upper surface 428 and the lower surface 430. The recessed area 560 at the intersection the annular frame member 416 and the flange 442 is circular for receiving the sonolucent upper wall 226.

Threaded attachment is facilitated by the provision of mating threads 432t on the exterior side wall 432 of the sonolucent upper wall 426 and the circumferential internal surface 560t of the recessed area 560. While threads are disclosed in accordance with this embodiment, it is appreciated other selectively releasably coupling mechanisms are known, for example, bayonet type connection, press fit connections, etc., that could be used in conjunction with the present invention. Further, screws could be used as is disclosed above with reference to the various embodiments.

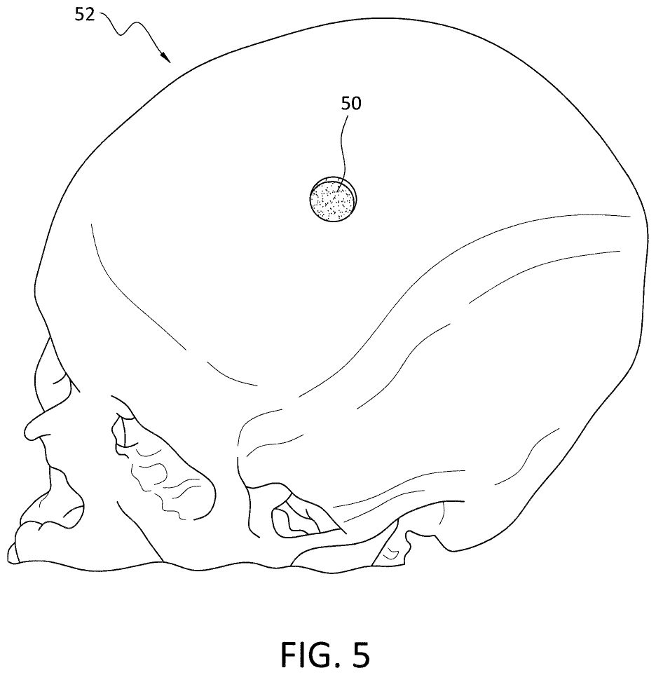

In practice, and with reference to FIGS. 5, 6, and 7, a cranial defect 50 the size of the cranial reconstruction device 10 is formed in the cranium 52. Thereafter, the cranial reconstruction device 10 is positioned and secured within the cranial defect and an ultrasound transducer 60 may be used in conjunction with the cranial reconstruction device 10. In particular, the tubular base member of the cranial reconstruction device 10 is implanted within the cranial defect 50 such that the cranial reconstruction device covers the entire internal surface of the cranial defect thereby preventing bone growth within the aperture defined by the cranial defect 50.

In accordance with a disclosed embodiment, an ultrasound transducer 60 optimized for use in conjunction with the cranial reconstruction device 10 is also provided. The ultrasound transducer 60 is optimized for imaging and interacting with the neuroanatomy, specifically the brain. The ultrasound transducer 60 is pre-set to frequencies, gain, field of view, etc. that are optimal for brain imaging through the present cranial reconstruction device 10.

Because the attenuation of ultrasound signals passing through the cranial reconstruction device 10 is known, this information may be used to optimize imaging. For example, and considering a cranial reconstruction device 10 composed of clear sonolucent PMMA and having a sonolucent upper wall 26 with a thickness less than 3.5 mm, the cranial reconstruction device 10 exhibits attenuation characteristics resulting in minimal degradation of the ultrasonic waves generated by the ultrasound transducer 60.

As those skilled in the art will appreciate, the amplitude change of a decaying plane wave can be expressed as:

A=A₀e^{αd}

where,
A₀ is the unattenuated amplitude of the propagating wave
A is the reduced amplitude after the wave has traveled a distance d
α is the attenuation constant measured in nepers/length (wherein Np/m may be converted to decibels by dividing α by 0.1151), where a neper is a dimensionless quantity
e is the exponential (or Napier's constant) which is equal to approximately 2.71828.

It is further appreciated attenuation of an ultrasonic wave is generally a function of the frequency of the ultrasonic wave and intrinsic property of the medium. This is used to differentiate PMMA from other implant materials. It is further appreciated that Attenuation Coefficient as measured in dB/(mHz×cm) represents the intrinsic property of a medium to attenuate sound waves at a given frequency. With the foregoing in mind the cranial reconstruction device 10, in particular the sonolucent upper wall 26 of the cranial reconstruction device 10, exhibits attenuation of no more than 6 dB/cm at frequencies between 1 MHz and 9 MHz. Within the range of 2 MHz to 2.5 MHz, the cranial reconstruction device 10 exhibits even better (that is, lower) attenuation characteristics.

Using this information, the frequency and gain of the ultrasound transducer 60 is varied to achieve imaging goals. For example, and in accordance with one embodiment, the ultrasound transducer 60 applies a frequency and gain resulting in a wide field of view allowing for the identification of various neural anatomy landmarks. With the landmark information from the initial scan in hand, the ultrasound transducer 60 is operated in doppler ultrasound mode to identify various vascular elements of interest. The image data from the initial scan and the doppler ultrasound scan is then processed and the information is used to develop a generated neural anatomy landscape. With the neural anatomy landscape in mind, the frequency and gain of the ultrasound transducer 60 are adjusted to allow the ultrasound transducer 60 to image highly specific parts of the neural anatomy. In this way, the ultrasound transducer 60 of the present invention is capable of identifying and imaging specific neural anatomy as desired.

For example, and considering an exemplary embodiment for relative deep imaging in the depth range of 9-15 cm, a sector array (or phased array) transducer probe is utilized with the abdominal factory preset, a frequency of approximately 1 MHz to approximately 3 MHz, a dynamic range of approximately 55 dB to approximately 65 dB, a gain of approximately 55 dB to approximately 70 dB. Other parameters that may be varied include time gain compensation, ultrasound Gray Map setting, line density, frame average persistence, edge enhancement, Doppler gain, pulse repetition period (PRF), wall filter, imaging angle, focal zone (wherein a single focus point is to be place at or slightly below the region of interest and this maintains the highest frame rate capability of the system (additional focus zones may be added to improve resolution within a larger region but doing so will decrease the frame rate)), etc. An additional consideration for use in conjunction with ICP (Intracranial Pressure) monitoring is an advanced feature of ultrasound called “shear wave elastography.” Those skilled in the art will appreciate that these parameters are dependent upon the manufacturing characteristics of the specific ultrasound system being used. It is also appreciated that scan parameters will be adjusted to accommodate ambient light.

Considering an exemplary embodiment for more superficial imaging in the depth range of approximately 3 cm, a linear transducer probe is utilized with the “vascular access” or “upper extremity vascular” factory preset, at frequency of approximately 12 MHz to approximately 15 MHz. As with the prior embodiment, parameters may be varied depending upon the manufacturing characteristics of the specific ultrasound system being used.

Further still, the ultrasound transducer may be optimized with or without the use of contrast agents to measure cerebral blood flow, intra-cranial pressure, identify tumors (or their resection plane), measure ventricles, or with or without a combination of non-imaging ultrasound functions such as dilating the BBB, Stimulating the brain (ala Deep Brain Stimulation), or ablating/cavitating brain tissue.

Considering the implementation of the ultrasound transducer in conjunction with contrast agents, the utilization of ultrasound with the sonolucent cranial reconstruction device 10 of the present invention allows one to act upon and/or monitor a contrast agent as it travels within the brain. This allows for various applications, including, but not limited to,
Imaging confirmation of blood flow immediately after surgery,
Imaging confirmation of blood flow during follow-up examinations,
Imaging confirmation of blood flow to desired locations within the brain,
Imaging visualization of, and guidance during, interventional procedures to guide a wire and/or catheter through the neurovasculature to a desired location in the brain, and to provide an image while a procedure is performed through the catheter, for example, placing a coil within the brain to treat aneurysms, and
activation of a contrast agent as it passes a particular point of interest and is subjected to ultrasound for activation thereof, for example, where the contrast agents are in the form of microbubbles.

Further still, the ultrasound transducer may be used in conjunction with drug delivery by
subjecting a drug to ultrasound at a specific location within the brain so that the drug is active at a highly specific location, or to enhance the effectiveness of the drug through delivery of ultrasound, regardless whether the drug is delivered by direct injection into the brain or is introduced indirectly such as parentally or through an endovascular catheter introduced to a desired location in the brain for this purpose,
subjecting the drug to ultrasound at a specific location within the brain to assist the drug in passing through the brain blood barrier, such as by causing brain cells to open up to permit larger sized molecules to pass, and
therapeutically releasing nano-encapsulated drugs through the application of ultrasound wherein the ultrasound breaks down the encapsulation material to release the drug.

It is contemplated such drug delivery mechanisms would find applications in therapies where delivery of a drug directly to the brain might minimize side effects and enhance the overall effectiveness of the drug, for example, in the treatment of Parkinson's, Alzheimer's, etc.

Introducing drugs in this manner allows for one to only worry about the drug reaching the treatment area. Introducing drugs in this manner also allows for intermittent dosing as the drug may be selectively activated upon the application of ultrasound, which may be controlled in a highly efficient and effective manner either through an implanted or handheld ultrasound transducer. Similarly, implementation of an ultrasound transducer array in conjunction with the cranial reconstruction device 10 would also allow for the application of ultrasound in different areas of the brain so that the location of treatment may be adjusted.

While the preferred embodiments have been shown and described, it will be understood that there is no intent to limit the invention by such disclosure, but rather, is intended to cover all modifications and alternate constructions falling within the spirit and scope of the invention.

1. A sonolucent cranial reconstruction device optimized for trans-cranioplasty ultrasound, comprising:
a tubular base member and a sonolucent upper wall extending across a top of the tubular base member, the tubular base member being shaped and dimensioned for positioning within an aperture defined by a cranial defect such that bone growth is prevented within the aperture defined by the cranial defect.

2. The sonolucent cranial reconstruction device according to claim 1, wherein the tubular base member extends a thickness of a cranium to mechanically prevent bone remodeling in the cranial defect, thereby preserving acoustic pass-through.

3. The sonolucent cranial reconstruction device according to claim 1, wherein the tubular base member is formed with a cross sectional profile selected from the group consisting of cylindrical, square, rectangular, oval, and polyhedral.

4. The sonolucent cranial reconstruction device according to claim 1, wherein the tubular base member includes an annular frame member defining an outer perimeter of the tubular base member, the annular frame member includes an upper surface, a lower surface, an inner sidewall extending between the upper surface and the lower surface along a cavity defined by an interior of the annular frame member, and an outer sidewall extending between the upper surface and the lower surface of the annular frame member.

5. The sonolucent cranial reconstruction device according to claim 4, wherein the inner sidewall and the outer sidewall define consistent and continuous diameters.

6. The sonolucent cranial reconstruction device according to claim 4, wherein the tubular base member is 7 mm to 9 mm from the upper surface to the lower surface.

7. The sonolucent cranial reconstruction device according to claim 4, wherein the sonolucent upper wall is coextensive with the upper surface of the annular frame member such that the sonolucent upper wall and the inner sidewall define a cavity of the cranial reconstruction device.

8. The sonolucent cranial reconstruction device according to claim 4, wherein a thickness of the sonolucent upper wall is less than a length of the tubular base member, thus forming the cavity defining an open area inside the tubular base member.

9. The sonolucent cranial reconstruction device according to claim 8, wherein a space defined by the cavity is filled with a highly ultrasound transmissive fluid, gel, or other material to enhance or otherwise control passage of ultrasound waves through the cranial reconstruction device.

10. The sonolucent cranial reconstruction device according to claim 8, wherein a lensing window is integrated into the cavity of the tubular base member to manipulate or alter ultrasound wave.

11. The sonolucent cranial reconstruction device according to claim 1, wherein the sonolucent upper wall includes an upper surface, a lower surface, and a sidewall extending between the upper surface and the lower surface.

12. The sonolucent cranial reconstruction device according to claim 11, wherein the sidewall of the sonolucent upper wall is tapered inwardly along a lower portion thereof such that a diameter of the sidewall decreases as it extends from the upper surface toward the lower surface of the sonolucent upper wall.

13. The sonolucent cranial reconstruction device according to claim 11, wherein the sonolucent upper wall is further provided with outwardly extending tab members coextensive with the sonolucent upper wall.

14. The sonolucent cranial reconstruction device according to claim 1, wherein the tubular base member and the sonolucent upper wall are integrally formed.

15. The sonolucent cranial reconstruction device according to claim 14, wherein the tubular base member and the sonolucent upper wall are manufactured from sonolucent poly (methyl methacrylate) (PMMA).

16. The sonolucent cranial reconstruction device according to claim 1, wherein the sonolucent upper wall and the tubular base member are distinct pieces that are selectively assembled and/or disassembled.

17. The sonolucent cranial reconstruction device according to claim 16, wherein the tubular base member and the sonolucent upper wall are formed from different materials offering optimal physical characteristics for their intended purposes.

18. The sonolucent cranial reconstruction device according to claim 17, wherein the tubular base member is made of porous polyethylene.

19. The sonolucent cranial reconstruction device according to claim 17, wherein the sonolucent upper wall is made of PMMA.

20. The sonolucent cranial reconstruction device according to claim 1, further including an ultrasound transducer.

21. A method for preventing bone growth within a cranial defect, comprising:
forming a cranial defect;
implanting a cranial reconstruction device within the cranial defect such that the cranial reconstruction device covers the entire internal surface of the cranial defect.

22. The method according to claim 21, wherein the cranial reconstruction device is sonolucent.

23. The method according to claim 22, further including a step of passing ultrasound through the cranial reconstruction device.

24. The method according to claim 22, wherein the cranial reconstruction device includes a tubular base member and a sonolucent upper wall extending across a top of the tubular base member.

25. The method according to claim 22, further including a step of acting upon and/or monitoring a contrast agent as it travels within the brain.

26. The method according to claim 25, further including a step of imaging confirmation of blood flow immediately after surgery, imaging confirmation of blood flow during follow-up examinations, imaging confirmation of blood flow to desired locations within the brain, imaging visualization of, and guidance during, interventional procedures to guide a wire and/or catheter, or activating a contrast agent as it passes a particular point of interest and is subjected to ultrasound for activation thereof.

27. The method according to claim 22, further including a step of utilizing ultrasound in conjunction with drug delivery.

28. The method according to claim 27, further including a step of subjecting a drug to ultrasound at a specific location within the brain so that the drug is active at a highly specific location, subjecting a drug to ultrasound at a specific location within the brain to enhance effectiveness of the drug through delivery of ultrasound, subjecting the drug to ultrasound at a specific location within the brain to assist the drug in passing through the brain blood barrier, or therapeutically releasing nano-encapsulated drugs through the application of ultrasound wherein the ultrasound breaks down the encapsulation material to release the drug.
